(19) United States
(12) Patent Application Publication
(10) Pub. No.: US 2026/0207285 A1
(43) Pub. Date: Jul. 23, 2026

(54) ENDOLUMINAL INSTRUMENT CONSTRAINER

(71) Applicant: Auris Health, Inc., Santa Clara, CA (US)

(72) Inventors: Nicholas J. Eyre, Sunnyvale, CA (US); Bryan James Culver, Mountain View, CA (US); Jingyi Xu, Mountain View, CA (US); Samuel J. Malanowski, Portland, OR (US)

(21) Appl. No.: 19/568,069

(22) Filed: Mar. 16, 2026

Related U.S. Application Data
(63) Continuation of application No. PCT/IB2025/059452, filed on Sep. 19, 2025.
(60) Provisional application No. 63/707,699, filed on Oct. 15, 2024.
(60) Provisional application No. 63/878,431, filed on Sep. 9, 2025.

(51) Int. Cl. A61B 34/37; A61B 34/30; A61M 25/01

(52) U.S. Cl. CPC A61B 34/37; A61B 2034/301; A61M 25/0113

(57) ABSTRACT

Among other things, a robotic medical system is disclosed. The robotic medical system includes an instrument handle and a flexible elongate instrument extending distally from the instrument handle for insertion into a patient at an insertion site. The instrument handle is configured to apply torque to the flexible elongate instrument for rolling the flexible elongate instrument about a roll axis. The flexible elongate instrument is configured to hang between the instrument handle and the insertion site to define a service loop. The robotic medical system also includes a planar constrainer extending along the service loop. The planar constrainer is configured to inhibit movement of the service loop out of a plane.

### PRIORITY

This application claims the benefit of U.S. Pat. App. No. 63/878,431, entitled “Endoluminal Instrument Constrainer,” filed Sep. 9, 2025, and U.S. Pat. App. No. 63/707,699, entitled “Endoluminal Instrument Constrainer,” filed Oct. 15, 2024, the disclosures of which are incorporated by reference herein.

### BACKGROUND

Minimally invasive medical procedures, such as endoscopy or robotically-assisted surgery, are increasingly used for the diagnosis or treatment of a variety of patient conditions. These techniques are attractive for their potential to minimize trauma to the patient, reduce recovery times, enhance surgeon precision, or facilitate new surgical approaches that may not be possible with traditional technologies. Minimally invasive procedures often involve insertion of elongate instruments into a patient's body through small anatomical openings, such as natural orifices or small incisions. These instruments are then advanced to an anatomical site and used to observe, manipulate, or interact with tissue or objects within the patient. To allow advancement of these instruments through one or more anatomical passageways (e.g., a lumen) of the patient to the anatomical site, some such instruments may include a flexible shaft.

### BRIEF DESCRIPTION OF DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts an example of a medical system.

FIG. 2 depicts an example of a medical system.

FIG. 3 depicts a perspective view of an example of a medical system having a planar constrainer.

FIG. 4 depicts a perspective view of the planar constrainer of FIG. 3 coupled to an instrument handle of the medical system.

FIG. 5 depicts a front elevation view of a linkage of the planar constrainer of FIG. 3.

FIG. 6 depicts a perspective view of a pair of links of the linkage of FIG. 5.

FIG. 7A-7C each depict a side elevation view of the medical system of FIG. 3, where FIG. 7A depicts the planar constrainer in an expanded state, FIG. 7B depicts the planar constrainer in a partially compressed state, and FIG. 7C depicts the planar constrainer in a fully compressed state.

FIGS. 8A-8B each depict a side elevation view of an example of a medical system having a flexible elongate instrument and a Bowden-type feed assembly, where FIG. 8A depicts the flexible elongate instrument in a retracted state, and FIG. 8B depicts the flexible elongate instrument in an advanced state.

FIGS. 9A-9B each depict a side elevation view of an example of a medical system having a flexible elongate instrument, a Bowden-type feed assembly, and a planar constrainer, where FIG. 9A depicts the flexible elongate instrument in a retracted state, and FIG. 9B depicts the flexible elongate instrument in an advanced state.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description and appended drawings contain certain examples and configurations of this technology and are not intended to be an exhaustive disclosure of the only configurations in which the technology may be practiced. Other examples, features, aspects, embodiments, and advantages of the technology will be apparent to those skill in the art from this disclosure. As will be realized, the technology described herein is capable of other different and obvious aspects, all without departing from the inventive concepts disclosed herein. Accordingly, the drawings and descriptions should be regarded as illustrative in nature and not restrictive. In some instances, well-known structures and components are not described in detail or are shown in block diagram form to avoid obscuring concepts of this technology.

As noted above, minimally invasive procedures can involve insertion of elongate instruments through small anatomical openings, such as natural orifices or small incisions. To allow advancement of these instruments through one or more anatomical passageways (e.g., a lumen) of the patient to the anatomical site, some such instruments may include a flexible elongate instrument. In some procedures, it may be desirable to roll the instrument about a roll axis.

Among other things, the following description relates to technologies that can facilitate set up, stabilization, or control of robotic instrumentation in medical procedures. In illustrated examples, a planar constrainer is depicted that can be mounted to a robot along a flexible elongate instrument for inhibiting out-of-plane movement of the instrument to thereby promote desired rolling of the instrument about the roll axis. In other illustrated examples, a feed band is depicted that can insert a flexible elongate instrument into a patient without requiring feed roller wheels or other bulky components at or near the insertion site.

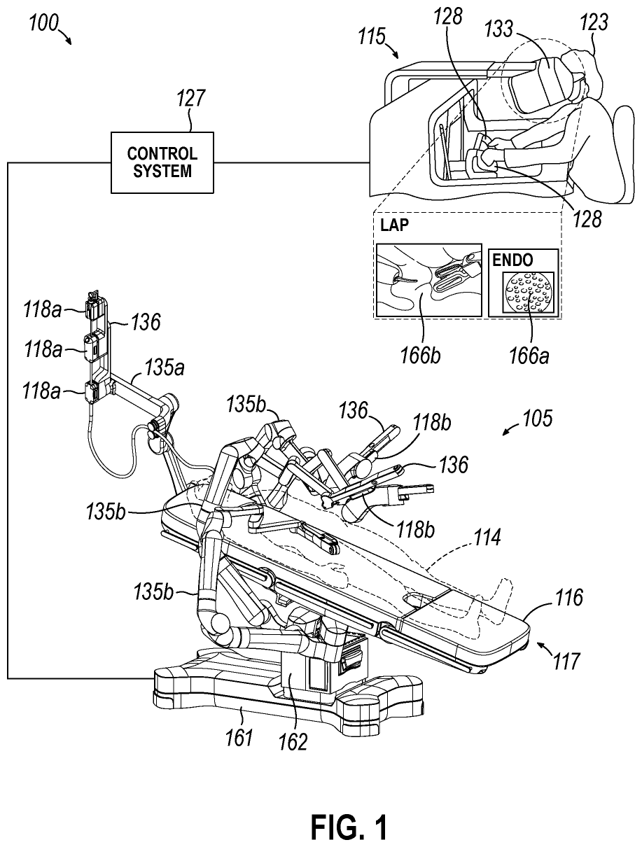

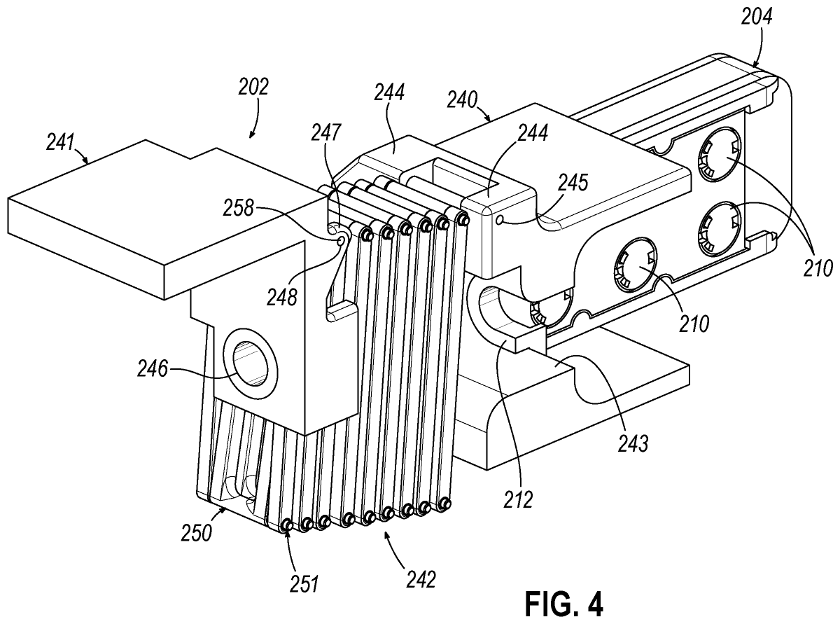

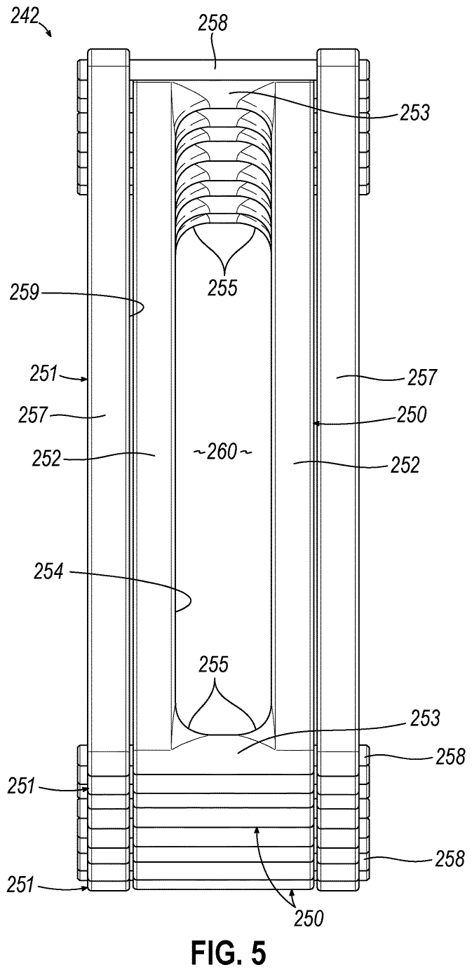

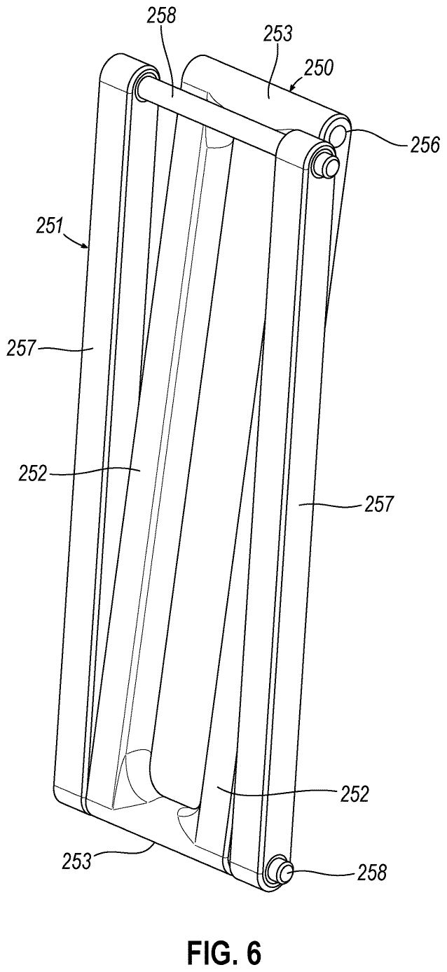

These and other features of this technology are further described below with respect to examples of FIGS. 1-9B. However, there are multiple inventive concepts disclosed herein which may be practiced independently, in combination, or in other contexts beyond these particular examples. Accordingly, these examples are explanatory in nature but should not be construed as limiting.

FIG. 1 depicts an example of a surgical system (or “medical system”), in accordance with some embodiments. The surgical system 100 is configured as a robotic system that can facilitate robotically-assisted procedures. The surgical system 100 can, for example, be configured to perform endoscopic procedures, laparoscopic procedures, combined endoscopic and laparoscopic procedures, and/or other types of procedures for purposes of diagnosis and/or treatment.

Surgical system 100 includes a surgical robot 105 having one or more robotic manipulators 135 configured to manipulate one or more instruments 118. These instruments 118 may be inserted into a patient 114 through one or more anatomical openings, such as natural orifices or incisions, to perform various tasks. In the illustrated configuration, multiple robotic manipulators 135 are shown, where each robotic manipulator 135 controls one or more instruments 118 respectively mounted thereto. The robotic manipulator 135 can include a robotic arm having a series of links connected by a series of joints to form a kinematic chain that can be actuated to various poses to control positioning of the instrument(s) 118. Instruments 118 can be mounted to an instrument holding portion 136 arranged at a distal end of the robot arm. Each instrument holding portion 136 can include a tool driver housing one or more actuators (e.g., motors or actuator stacks) for controlling mechanisms within instruments mounted thereto. The surgical system 100 can be configured to manipulate a variety of instruments or perform a variety of tasks in various configurations. Examples of instruments include endoscopes, sheaths, graspers, needle drivers, energy instruments, biopsy instruments, forceps, snares, and various elongate instruments having flexible or rigid shafts that may be inserted into the patient's body. Examples of surgical tasks include grasping tissue, excising tissue, capturing images, delivering energy, suturing, stapling, and otherwise manipulating or observing tissue or objects within the patient.

As seen in FIG. 1, surgical robot 105 is coupled to, or integrated with, a surgical table 117 (e.g., an operating table). Surgical table 117 includes a patient platform 116 (e.g., a table top) that supports a patient 114 positioned thereon. Patient platform 116 is coupled to or supported by a column 162, which is coupled to or supported by a base 161. The base 161 can rest on a floor of the procedure area (e.g., an operating room). In the illustrated example, patient platform 116 can be actuated to tilt or adjust an orientation of the patient platform 116 (or patient 114) relative to the base 161 or operating room floor. The surgical table 117 also provides a base for mounting the robotic manipulators 135, which are coupled to the surgical table 117 via an arm support 163.

Surgical robot 105 can be controlled or actuated based on commands received from a physician console 115 (e.g., telemanipulated). Physician console 115 includes or is coupled to one or more input devices 128, which user 123 can operate to provide commands for teleoperation of robot 105 or to control various aspects of the system. As illustrated, the physician console 115 includes a pair of handheld input devices, including a left-hand input device and a right-hand input device. Each of the handheld input devices can include, for example, a gimbal that provides various degrees of freedom for movement or manipulation of the input device 128 in space to control a corresponding position or orientation of an instrument 118. Although gimbal-based handheld input devices are shown in FIG. 1, in various configurations, the physician console 115 can additionally or alternatively include a variety of types of input devices. Examples of input devices include handheld pendants, touch screens, touch sensors, foot pedals, buttons, joysticks, trackballs, keyboards, mice, or human interface devices that can receive input from one or more users.

As seen in FIG. 1, physician console 115 can also include a display 133, which can be configured to present images for observation by the user 123. For example, display 133 can be configured to display endoscopic images 166 captured with the instruments 118, so that user 121 can provide commands to the robot 105 via the input device 130 while viewing a real-time camera feed captured within the patient's anatomy. Alternatively, or in combination, display 133 can be configured to present, for example, pre-operative images, navigation information, and/or interactive menus. In the illustrated example, display 133 is configured as an immersive viewer, where the user 123 may insert their head into the viewer for an immersive three-dimensional (e.g., stereoscopic) image. Alternatively, or combination, the surgeon console may include an open display, such a flat panel display screen that provides two-dimensional and/or three-dimensional images. Alternatively, or in combination, one or more displays 133 can be provided on devices such as the surgical robot 105 itself, a pendant, a support tower (e.g., a vision processing tower, fluidics tower, and/or energy deliver tower).

Control system 127 includes processing circuitry and memory communicatively coupled to the robot 105 and/or physician console 115. Control system 127 can be configured to implement functions of the surgical system 100, such as controlling or actuating the robot 105, controlling or operating the instruments 118, or processing inputs and/or outputs to or from physician console 115. For example, processing circuitry of the control system 127 can be configured via hardware or software programming to implement any functions described further herein in connection with operation of the surgical system 100. Examples of processing circuitry include one or more central processing units (CPUs), graphics processing units (GPUs), field programmable gate arrays (FPGAs), application specific integrated circuits (ASICs), or other processors configured to process inputs or outputs for the surgical system 100. As used herein, the term “processor” can encompass a single processing chip or integrated circuit, or multiple processing chips or integrated circuits that may be co-located or distributed in different locations and configured to execute functions described herein. Memory can store instructions that, when executed by the processor, cause execution of methods described herein. As used herein, the term “memory” can encompass any suitable non-transitory computer readable medium embodied in one or several memory devices, such as hard drives, flash memory, solid state memory, storage discs, or tapes. Components of the control system 127 may be physically disposed in or connected to components of the surgical system 100, such as the surgical robot 105 or the physician console 115, or components of the control system 127 may be otherwise communicatively coupled to components of the medical system 100 via various wired or wireless interconnections.

Although an example configuration of the surgical system 100 is shown and described, the surgical system 100 may be configured in various ways, where such variations are not all separately illustrated or explicitly enumerated for the sake of clarity and conciseness of this description. For example, while the example shown depicts robot 105 as table-based robot, in some configurations, robot 105 can be configured as a cart-based robot, where one or more robotic manipulators are coupled to a robotic cart that can be positioned beside the surgical table 117. Additionally, or alternatively, robot 105 can be configured as a boom-based robot, where robotic manipulators descend from an overhead boom that can be suspended above the patient, from a boom-based cart or from a ceiling of the operating room. In some variations, the surgical system 100 can include one or multiple robots 105 or robotic carts, where multiple robots or robotic carts may be configured to operate in cooperation with each other, or where a cart-based robot and a table-based robot are configured to operate in cooperation with each other. In some configurations, a robot 105 may include one or multiple robotic manipulators 135 to manipulate one or multiple instruments. For example, robot 105 can include one, two, three, four, five, six, or more robotic manipulators 135, and each manipulator can manipulate one or multiple instruments.

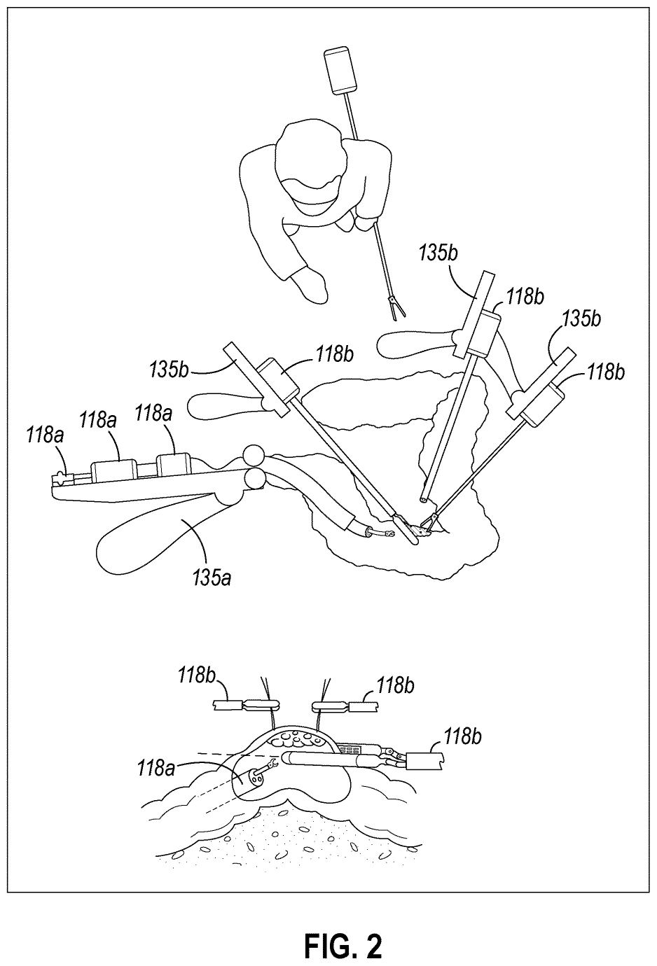

In some variations, as seen in FIGS. 1-2, surgical system 100 is configured to facilitate combined endoscopic and laparoscopic surgery (CELS). The robotic manipulator(s) 135 can include an endoscopic manipulator 135a (sometimes referred to herein as an “endodriver”), configured to manipulate one or more endoscopic (e.g., endoluminal) instruments 118a having flexible shafts, and one or more laparoscopic manipulators 135b (e.g., four laparoscopic manipulators) configured to manipulate laparoscopic instruments 118b having rigid shafts. In some instances, the endoscopic instruments can include a flexible endoscope to capture endoscopic images 166a, and the laparoscopic instrument can include a rigid laparoscope to capture laparoscopic images 166b. These images can be presented to the user (e.g., physician) 123 in concert with each other on display 133 to facilitate cooperative control of these instruments to perform surgical tasks.

Various technical concepts are described herein with reference to example configurations involving robotically-assisted endoscopy, laparoscopy, CELS, and gastrointestinal (GI) procedures (upper or lower). However, it will be appreciated that certain principles of the technology described herein can be applied in a variety of medical procedures or contexts. Some examples of procedures include bronchoscopy, ureteroscopy, laparoscopy, colonoscopy, gastrointestinal procedures, cardiovascular procedures, general surgery, or endoluminal surgery.

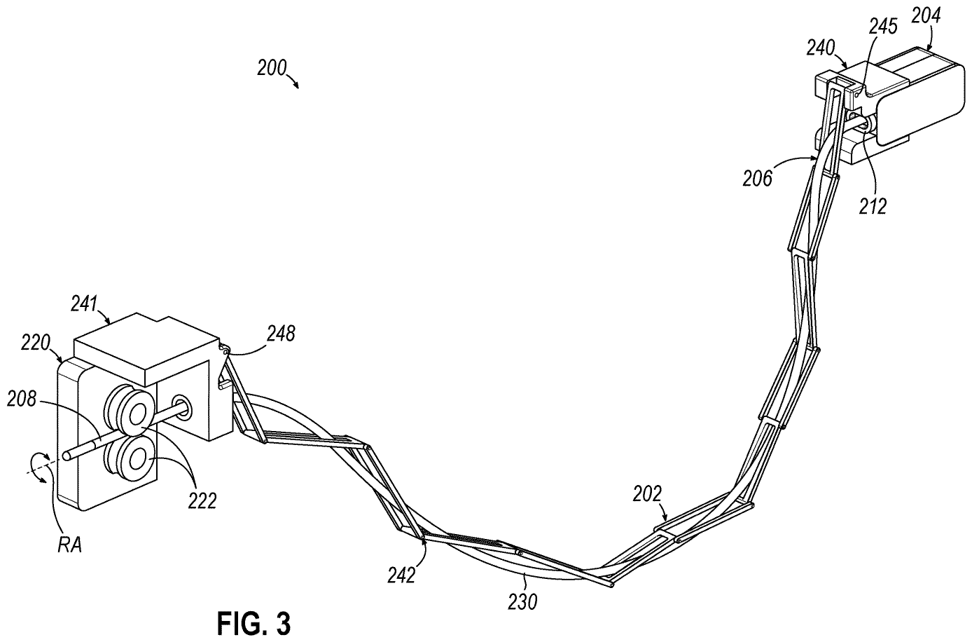

FIG. 3 depicts an example of a medical system (200) having a planar constrainer (202), in accordance with some embodiments.

As illustrated, medical system (200) includes an instrument handle (204) and a flexible elongate member (206) extending distally from instrument handle (204) to a distal end (208). Instrument handle (204) may be used to longitudinally translate (e.g., advance and retract) and/or steer (e.g., articulate) elongate member (206). By way of example only, elongate member (206) may include a scope, a sheath (e.g., an access sheath), a catheter, a shaft of a surgical tool (e.g., a biopsy tool) which may further include an end effector (not shown) at distal end (208) of elongate member (206), and/or any other suitable kind of elongate instrument.

Instrument handle (204) is configured to dock to an instrument holding portion such as that shown in FIG. 1 (e.g., an instrument device manipulator (IDM) or tool driver). In the example shown, instrument handle (204) includes a plurality of (e.g., six) rotatable drive inputs (210) configured to engage corresponding drive outputs of such an IDM. Instrument handle (204) also includes a distal port (212) that is configured to slidably receive elongate member (206) for permitting passage of elongate member (206) distally out of an interior of instrument handle (204). In some cases, at least some drive inputs (210) may be operatively coupled to distal end (208) of elongate member (206) via corresponding pull wires (not shown), such that these drive inputs (210) may be configured to selectively deflect distal end (208) of elongate member (206) laterally and/or vertically away to thereby steer elongate member (206). For example, a first pair of drive inputs (210) may be configured to cooperate with each other to facilitate lateral deflection of distal end (208) while a second pair of drive inputs (210) may be configured to cooperate with each other to facilitate vertical deflection of distal end (208); and as another example, the first and second pairs of drive inputs (210) may be configured to cooperate with each other to facilitate advancement and retraction of at least a proximal portion of elongate member (206). In addition, or alternatively, one or both drive inputs (210) of a third pair of drive inputs (210) may be configured to facilitate rolling of elongate member (206) about a roll axis (RA) extending along a length of elongate member (206). For example, one or both drive inputs (210) of the third pair of drive inputs (210) may be operatively coupled with at least one roller wheel (not shown) configured to rotate about an axis parallel to the roll axis (RA) while frictionally engaging elongate member (206), such that rotation of the at least one roller wheel driven by the one or both drive inputs (210) of the third pair of drive inputs (210) may apply torque to elongate member (206) and thereby cause rolling of elongate member (206) about the roll axis (RA). While such rolling of elongate member (206) about the roll axis (RA) may be initiated via application of torque to elongate member (206) at instrument handle (204), it will be appreciated that the body of elongate member (206) may have sufficient material strength to transmit such torque distally along the length of elongate member (206) all the way to distal end (208) to thereby cause rolling of distal end (208).

In the example shown, medical system (200) also includes an elongate member feed assembly (220) positioned distally of instrument handle (204), such as at or near an insertion site of elongate member (206) into the patient, and configured to facilitate advancement and retraction of at least a distal portion of elongate member (206). In this regard, elongate member feed assembly (220) of the present example includes a pair of roller wheels (222) spaced apart from each other to receive elongate member (206) therebetween, and configured to rotate about respective axes perpendicular to the roll axis (RA) while frictionally engaging elongate member (206), such that rotation of one or both roller wheels (222) may cause advancement or retraction of elongate member (206). In some cases, one roller wheel (22) may be operatively coupled to a motor (not shown) and thus may be referred to as a driven wheel, while the other roller wheel (222) may not be operatively coupled to such a motor but rather may be configured to rotate about the respective axis in response to the translation of elongate member (206) that is caused by rotation of the driven wheel (222), and thus may be referred to as an idler wheel (222). In some other cases, both roller wheels (222) may be driven wheels (222).

As shown, a portion of elongate member (206) extends between instrument handle (204) and elongate member feed assembly (220), and may have a length greater than the distance between instrument handle (204) and elongate member feed assembly (220) to define a service loop (230). As noted above, instrument handle (204) may be configured to advance and retract at least a proximal portion of elongate member (206), while elongate member feed assembly (220) may be configured to advance and retract at least a distal portion of elongate member (206). For example, instrument handle (204) may be configured to advance a portion of elongate member (206) that is proximal of instrument handle (204) into service loop (230), and/or to retract at least a portion of service loop (230); while elongate member feed assembly (220) may be configured to advance at least a portion of service loop (230) into the patient, and/or to retract a portion of elongate member (206) that is distal of elongate member feed assembly (220) into service loop (230). It will be appreciated that providing service loop (230) between instrument handle (204) and elongate member feed assembly (220) may minimize the length of the portion of elongate member (206) that is proximal of instrument handle (204), thereby contributing to the compact configuration of medical system (200).

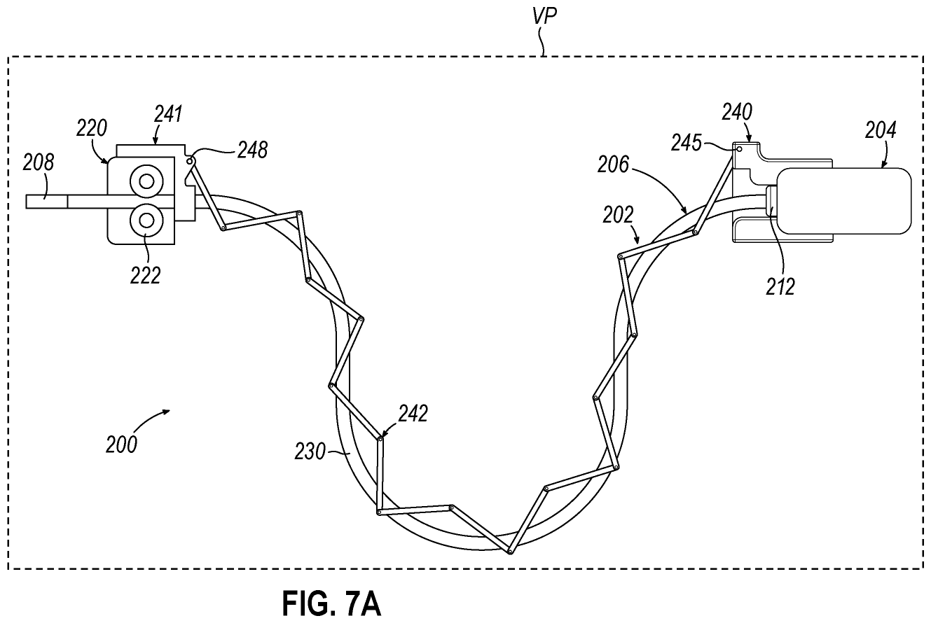

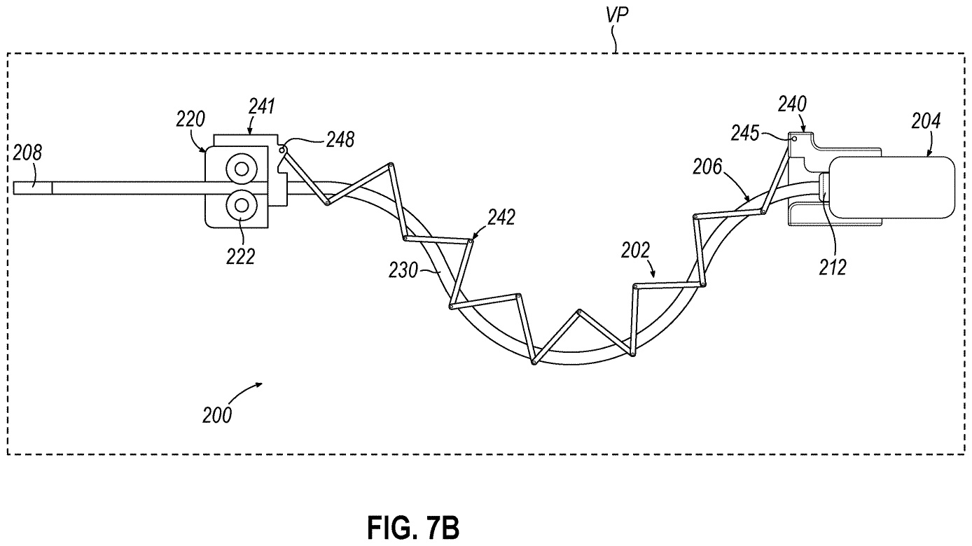

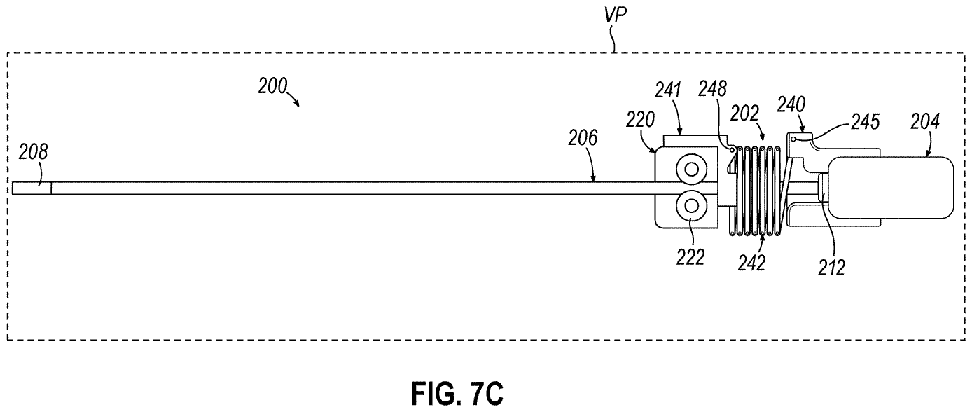

As shown, service loop (230) may hang (e.g., sag) freely under the influence of gravity between instrument handle (204) and elongate member feed assembly (220), such that service loop (230) may reside within a substantially vertical plane (VP) (e.g., defined by a line between instrument handle (204) and elongate member feed assembly (220)), as shown in FIGS. 7A-7C. Due to the resulting curvature of elongate member (206), at least a portion of the roll axis (RA) may be curved (e.g., along the length of service loop (230)).

As noted above, instrument handle (204) may be configured to initiate rolling of elongate member (206) about the roll axis (RA) by applying torque to elongate member (206), and the body of elongate member (206) may have sufficient material strength to transmit such torque distally along the length of elongate member (206) all the way to distal end (208). In some instances, the transmission of such torque along the length of elongate member (206) to distal end (208) may be resisted by external forces exerted upon a distal portion of elongate member (206). For example, the frictional engagement between elongate member (206) and one or both roller wheels (222) of elongate member feed assembly (220) may resist the transmission of such torque to distal end (208). As another example, any frictional engagement that might be present between elongate member (206) and the orifice or incision at the insertion site may resist the transmission of such torque to distal end (208). The resistance to the transmission of such torque to distal end (208) by external forces exerted upon a distal portion of elongate member (206) may be referred to as rolling resistance. In some cases, in response to the distal portion of elongate member (206) encountering such rolling resistance, service loop (230) may have a tendency to absorb at least a portion of the torque being transmitted distally along the length of elongate member (206) by moving (e.g., swinging) out of the vertical plane (VP). In other words, at least a portion of the torque being transmitted distally along the length of elongate member (206) may be converted into movement of service loop (230) out of the vertical plane (VP), rather than reaching distal end (208). Thus, distal end (208) may roll about the roll axis (RA) to a lesser extent than desired, or in some cases may even fail to roll about the roll axis (RA) at all.

In this regard, medical system (200) of the present example further includes planar constrainer (202) positioned along service loop (230) and configured to permit movement of service loop (230) within the vertical plane (VP) while inhibiting (e.g., preventing) movement of service loop (230) out of the vertical plane (VP). In other words, planar constrainer (202) is configured to constrain movement of service loop (230) to movements within the vertical plane (VP), such as distal movement (e.g., from instrument handle (204) toward elongate member feed assembly (220)) within the vertical plane (VP), proximal movement (e.g., from elongate member feed assembly (220) toward instrument handle (204)) within the vertical plane (VP), upward movement within the vertical plane (VP), and downward movement within the vertical plane (VP).

In the example shown, planar constrainer (202) includes a proximal handle adapter (240) configured to securely mount to instrument handle (204), a distal feed adapter (241) configured to securely mount to elongate member feed assembly (220), and an expandable linkage (242) extending distally from handle adapter (240) to feed adapter (241). Handle adapter (240) may be securely mounted to instrument handle (204) via any suitable fasteners (e.g., bolts, screws, etc.) for fixing handle adapter (240) against movement relative to instrument handle (204). Similarly, feed adapter (241) may be securely mounted to elongate member feed assembly (220) via any suitable fasteners (e.g., bolts, screws, etc.) for fixing feed adapter (241) against movement relative to elongate member feed assembly (220).

Handle adapter (240) of the present example includes a channel (243) configured to align with and/or receive distal port (212) of instrument handle (204) to thereby allow passage of elongate member (206) from instrument handle (204) to linkage (242). As shown, handle adapter (240) also includes a pair of distal walls (244) laterally spaced apart from each other to receive a proximal portion of linkage (242) therebetween. Handle adapter (240) further includes a pair of bores (245) extending laterally through respective distal walls (244) above channel (243) to facilitate pivotable coupling of the proximal portion of linkage (242) to handle adapter (240). In some versions, linkage (242) may be mounted directly to instrument handle (204), such that handle adapter (240) may be omitted.

Feed adapter (241) of the present example includes a low-friction sleeve bearing (246) configured to allow passage of elongate member (206) from linkage (242) to elongate member feed assembly (220). As shown, feed adapter (241) also includes a pair of proximal walls (247) (one shown) laterally spaced apart from each other to receive a distal portion of linkage (242) therebetween. Feed adapter (241) further includes a pair of bores (248) extending laterally through respective proximal walls (247) above bearing (246) to facilitate pivotable coupling of the distal portion of linkage (242) to handle adapter (240). In some versions, linkage (242) may be mounted directly to elongate member feed assembly (220), such that feed adapter (241) may be omitted.

Linkage (242) of the present example includes a plurality of links (250, 251) and, more particularly, includes a plurality of first links (250) and a plurality of second links (251) pivotably coupled to each other in a longitudinally-alternating arrangement. In the example shown, each first link (250) includes a pair of vertical bars (252) and a pair of horizontal bars (253) integrally formed together with each other as a unitary (e.g., monolithic) piece. Vertical bars (252) each have a first length, and horizontal bars (253) each have a second length substantially less than the first length, such that each first link (250) has a substantially rectangular shape. In this manner, bars (252, 253) of each first link (250) collectively define a corresponding aperture (254) that is elongate in the vertical direction and that is configured to slidably receive elongate member (206). More particularly, vertical bars (252) of each first link (250) are horizontally spaced apart from each other to define a width of the corresponding aperture (254) that is substantially equal to or only slightly greater than an external cross-dimension (e.g., diameter) of elongate member (206), such that each aperture (254) is configured to permit sliding of elongate member (206) in a direction substantially transverse (e.g., perpendicular) to aperture (254) and such that vertical bars (252) are configured to inhibit (e.g., prevent) lateral movement of elongate member (206) within aperture (254); and horizontal bars (253) of each first link (250) are vertically spaced apart from each other to define a height of the corresponding aperture (254) that is substantially greater than the external cross-dimension (e.g., diameter) of elongate member (206), such that each aperture (254) is also configured to permit sliding of elongate member (206) from one horizontal bar (253) toward the other horizontal bar (253).

Each first link (250) of the present example includes rounded internal corners (255) at the interfaces between vertical bars (252) and horizontal bars (253) for providing smooth contact with elongate member (206) (e.g., at the upper and lower bounds of each aperture (254)) to thereby reduce any frictional engagement between elongate member (206) and bars (252, 253). Thus, in the example shown, each aperture (254) has a substantially rounded rectangular shape. It will be appreciated that each aperture (254) may have any other suitable shape, such as a substantially circular shape or a substantially obround shape, for example. In some cases, each aperture (254) may have a substantially rectangular shape with sharp internal corners. Each first link (250) further includes upper and lower bores (256) extending laterally through respective horizontal bars (253) to facilitate pivotable coupling of each first link (250) to the adjacent second link(s) (251) and/or to one of the adapters (240, 241), as described in greater detail below.

In the example shown, each second links (251) includes a pair of vertical bars (257) and a pair of horizontal axle pins (258) fixedly secured to each other. Horizontal axle pins (258) are configured to pivotably couple each second links (251) to the adjacent first link(s) (250) and/or to one of the adapters (240, 241). For example, the upper horizontal axle pin (258) of at least some second links (251) may be received within the upper bore (256) of the distally-adjacent first link (250) to pivotably couple each such second links (251) to the distally-adjacent first link (250); and/or the lower horizontal axle pin (258) of at least some second links (251) may be received within the lower bore (256) of the proximally-adjacent first link (250) to pivotably couple each such second links (251) to the proximally-adjacent first link (250). In the example shown, the upper horizontal axle pin (258) of the distal-most second links (251) pivotably couples the distal-most second links (251) to feed adapter (241) via bores (248), and another upper horizontal axle pin (258) pivotably couples the proximal-most first link (250) to handle adapter (240) via bores (245).

Vertical bars (257) each have the first length described above, and horizontal axle pins (258) each have a third length substantially less than the first length, such that each second links (251) has a substantially rectangular shape. In this manner, bars (257) and axle pins (258) of each second links (251) collectively define a corresponding substantially rectangular aperture (259) that is elongate in the vertical direction and that is configured to slidably receive elongate member (206) and/or that is configured to at least partially receive the adjacent first link(s) (250). More particularly, vertical bars (257) of each first link (250) are horizontally spaced apart from each other to define a width of the corresponding aperture (259) that is substantially equal to or greater than an external cross-dimension (e.g., width) of the adjacent first link(s) (250), such that each aperture (259) is configured to permit at least partial nesting of the adjacent first link(s) (250) therein. In the example shown, apertures (254, 259) of first and second links (250, 251) are configured to align with each other to collectively define a passageway (260) along which elongate member (206) may extend.

Due to the pivotable couplings between adjacent first and second links (250, 251) provided by the corresponding axle pins (258), linkage (242) may be expandable from at least one compressed (e.g., collapsed) state to at least one expanded state; and may be compressible (e.g., collapsible) from the at least one expanded state to the at least one compressed state. As noted above, the aperture (259) of each second link (251) is configured to permit at least partial nesting of the adjacent first link(s) (250) therein, such that first links (250) may at least partially nest within second links (251) when linkage (242) is in the at least one compressed state to thereby reduce the length of linkage (242) when linkage (242) is in the at least one compressed state. In addition, since linkage (242) of the present example is generally unsupported along its length (e.g., between the upper horizontal axle pin (258) that pivotably couples the distal-most second links (251) to feed adapter (241), and the upper horizontal axle pin (258) that pivotably couples the proximal-most first link (250) to handle adapter (240)), linkage (242) may be capable of hanging (e.g., sagging) between instrument handle (204) and elongate member feed assembly (220). However, the pivotable couplings between adjacent first and second links (250, 251) provided by the corresponding axle pins (258) may be sufficiently strong to inhibit (e.g., prevent) lateral movement of links (250, 251) relative to each other and/or relative to adapters (240, 241).

Thus, when linkage (242) is positioned about service loop (230) such that elongate member (206) extends through each aperture (254, 259) of links (250, 251) along passageway (260), linkage (242) may reside within the same vertical plane (VP) as service loop (230), and may inhibit (e.g., prevent) movement of service loop (230) out of the vertical plane (VP) while accommodating movement of service loop (230) within the vertical plane (VP). For example, linkage (242) may be capable of conforming to the hanging of service loop (230) between instrument handle (204) and elongate member feed assembly (220); and may further be capable of conforming to vertical movement of service loop (230). As noted above, first links (250) may at least partially nest within second links (251) when linkage (242) is in the at least one compressed state to thereby reduce the length of linkage (242) when linkage (242) is in the at least one compressed state, such as when linkage (242) is in a fully compressed state. Such a reduction in the length of linkage (242) when linkage (242) is in the at least one compressed state may provide a corresponding reduction in the length of service loop (230) when linkage (242) is in the at least one compressed state, which may thereby provide an increase in the length of the portion of elongate member (206) that is distal of elongate member feed assembly (220). In other words, minimizing the length of service loop (230) via nesting of links (250, 251) when linkage (242) is in the fully compressed state may maximize the length of the portion of elongate member (206) that is distal of elongate member feed assembly (220) and thus maximize the extent to which distal end (208) may be advanced into the patient.

Referring now to FIGS. 7A-7C, in an example of a method of use, planar constrainer (202) may be positioned along service loop (230), with handle adapter (240) securely mounted to instrument handle (204), feed adapter (241) securely mounted to elongate member feed assembly (220), and linkage (242) positioned about service loop (230) such that elongate member (206) extends through channel (243) of handle adapter (240), through each aperture (254, 259) of links (250, 251), and through sleeve bearing (246) of feed adapter (241). Instrument handle (204) may initially be spaced proximally away from elongate member feed assembly (220), and the portion of elongate member (206) extending between instrument handle (204) and elongate member feed assembly (220) may have a length greater than the distance between instrument handle (204) and elongate member feed assembly (220) to define service loop (230); thus, service loop (230) and linkage (242) may hang (e.g., sag) between instrument handle (204) and elongate member feed assembly (220) within the vertical plane (VP), such that linkage (242) is in an expanded state, as shown in FIG. 7A. In some instances, instrument handle (204) may initiate rolling of elongate member (206) about the roll axis (RA) by applying torque to elongate member (206) while linkage (242) is in the expanded state shown in FIG. 7A; and planar constrainer (202) may promote the transmission of such torque by the body of elongate member (206) distally along the length of elongate member (206) all the way to distal end (208) by inhibiting (e.g., preventing) such torque from being converted into movement of service loop (230) out of the vertical plane (VP).

Elongate member feed assembly (220) may then advance a portion of service loop (230) into the patient, thereby decreasing the length of service loop (230) such that service loop (230) and linkage (242) raise vertically together within the vertical plane (VP) and such that linkage (242) compresses to a partially compressed state, as shown in FIG. 7B. As linkage (242) compresses to the partially compressed state, first links (250) may at least partially nest within second links (251). In some instances, instrument handle (204) may initiate rolling of elongate member (206) about the roll axis (RA) by applying torque to elongate member (206) while linkage (242) is in the partially compressed state shown in FIG. 7B; and planar constrainer (202) may promote the transmission of such torque by the body of elongate member (206) distally along the length of elongate member (206) all the way to distal end (208) by inhibiting (e.g., preventing) such torque from being converted into movement of service loop (230) out of the vertical plane (VP).

Elongate member feed assembly (220) may continue to advance more of service loop (230) into the patient and, in some cases, instrument handle (204) may move (e.g., translate) distally toward elongate member feed assembly (220), thereby further decreasing the length of service loop (230) such that service loop (230) and linkage (242) raise further vertically together within the vertical plane (VP) and such that linkage (242) compresses to a fully compressed state, as shown in FIG. 7C. As linkage (242) compresses to the fully compressed state, first links (250) may further nest within second links (251). In some instances, instrument handle (204) may initiate rolling of elongate member (206) about the roll axis (RA) by applying torque to elongate member (206) while linkage (242) is in the fully compressed state shown in FIG. 7C; and planar constrainer (202) may promote the transmission of such torque by the body of elongate member (206) distally along the length of elongate member (206) all the way to distal end (208) by inhibiting (e.g., preventing) such torque from being converted into movement of service loop (230) out of the vertical plane (VP).

While the distal end of planar constrainer (202) is mounted to elongate member feed assembly (220) via feed adapter (241) in the example shown, the distal end of planar constrainer (202) may alternatively be mounted to any other suitable component of medical system (200). In this regard, medical system (200) may include a port, such as an introducer, to facilitate introducing distal end (208) of elongate member (206) into an anatomical opening of the patient, and the distal end of planar constrainer (202) may be mounted to the introducer. For example, the distal end of planar constrainer (202) may be mounted to the introducer via an introducer adapter, which may be similar to feed adapter (241). As another example, linkage (242) may be mounted directly to the introducer.

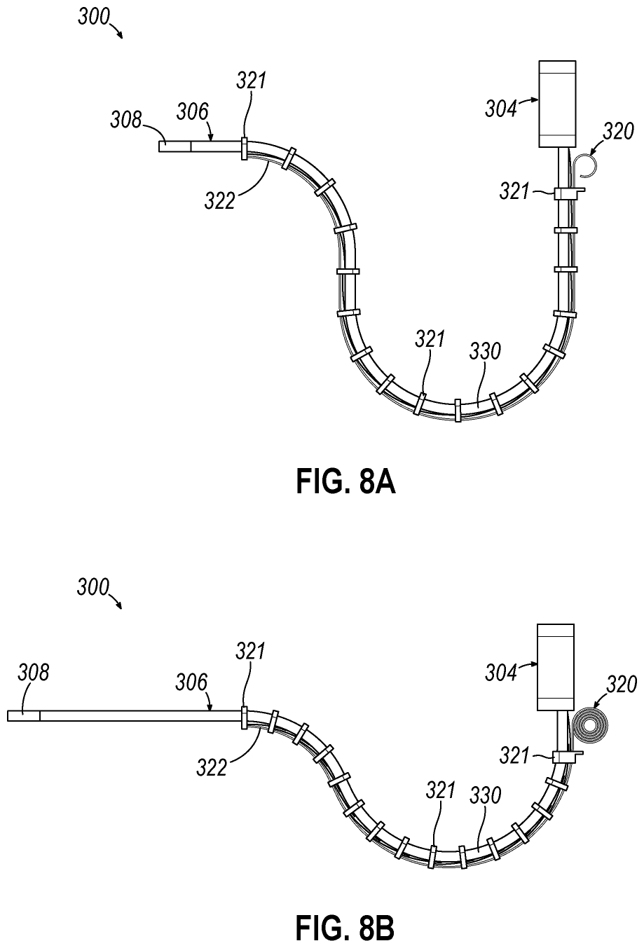

FIGS. 8A-8B depict an example of a medical system (300) having a Bowden-type elongate member feed assembly (320), in accordance with some embodiments.

As illustrated, medical system (300) includes an instrument handle (304) and a flexible elongate member (306) extending distally from instrument handle (304) to a distal end (308). Instrument handle (304) may be used to longitudinally translate (e.g., advance and retract) and/or steer (e.g., articulate) elongate member (306). By way of example only, elongate member (306) may include a scope, a sheath (e.g., an access sheath), a catheter, a shaft of a surgical tool (e.g., a biopsy tool) which may further include an end effector (not shown) at distal end (308) of elongate member (306), and/or any other suitable kind of elongate instrument.

Instrument handle (304) may include at least some drive inputs (not shown) operatively coupled to distal end (308) of elongate member (306) via corresponding pull wires (not shown), such that these drive inputs may be configured to selectively deflect distal end (308) of elongate member (306) laterally and/or vertically away to thereby steer elongate member (306) and/or to facilitate advancement and retraction of at least a proximal portion of elongate member (306). In addition, or alternatively, instrument handle (304) may include one or more drive inputs configured to facilitate rolling of elongate member (306) about a roll axis (RA) extending along a length of elongate member (306), such as in a manner similar to that described above.

In the example shown, medical system (300) also includes elongate member feed assembly (320) positioned distally of instrument handle (304), such as at or near an insertion site of elongate member (306) into the patient, and configured to facilitate advancement and retraction of at least a distal portion of elongate member (306). In this regard, elongate member feed assembly (320) of the present example includes a plurality of guides (321) slidably positioned along a portion of elongate member (306) that is distal of instrument handle (304); and a flexible feed band (322) routed along the same portion of elongate member (306) that guides (321) are positioned along. Feed band (322) may comprise a metallic material, for example. Feed band (322) of the present example is fixedly coupled to the distalmost guide (321) and slidably received by the other guides (321), and is configured to spool at or near instrument handle (304). In this regard, feed band (322) may be configured to be spooled onto a spindle (not shown) at or near instrument handle (304), and such a spindle may be operatively coupled to a motor (not shown) to facilitate spooling of feed band (322) onto the spindle and unspooling of feed band (322) from the spindle. In some versions, the distalmost guide (321) may be secured against movement relative to the insertion site, such that the distal end of feed band (322) may be stationary during spooling/unspooling of feed band (322). In addition, or alternatively, feed band (322) may be rotationally coupled to elongate member (306) (e.g., via one or more guides (321), such as the proximal-most guide (321)) such that feed band (322) may be configured to rotate together with elongate member (306) about the roll axis (RA).

As shown, a portion of elongate member (306) extends between instrument handle (304) and the distalmost guide (321) of elongate member feed assembly (320), and may have a length greater than the distance between instrument handle (304) and the distalmost guide (321) of elongate member feed assembly (320) to define a service loop (330). As noted above, instrument handle (304) may be configured to advance and retract at least a proximal portion of elongate member (306), while elongate member feed assembly (320) may be configured to advance and retract at least a distal portion of elongate member (306). For example, instrument handle (304) may be configured to advance a portion of elongate member (306) that is proximal of instrument handle (304) into service loop (330), and/or to retract at least a portion of service loop (330); while elongate member feed assembly (320) may be configured to advance at least a portion of service loop (330) into the patient, and/or to retract a portion of elongate member (306) that is distal of elongate member feed assembly (320) into service loop (330). It will be appreciated that providing service loop (330) between instrument handle (304) and elongate member feed assembly (320) may minimize the length of the portion of elongate member (306) that is proximal of instrument handle (304), thereby contributing to the compact configuration of medical system (300).

As shown, service loop (330) may hang (e.g., sag) freely under the influence of gravity between instrument handle (304) and the distalmost guide (321) of elongate member feed assembly (320), such that service loop (330) may reside within a substantially vertical plane (e.g., defined by a line between instrument handle (304) and the distalmost guide of elongate member feed assembly (320)). Since feed band (322) is routed along service loop (330) via guides (321), feed band (322) may reside within the same vertical plane as service loop (330).

As noted above, feed band (322) is configured to spool at or near instrument handle (304) while the distal end of feed band (322) is configured to be stationary, such that spooling of feed band (322) may cause advancement of elongate member (306) by decreasing the length of service loop (330) to thereby cause elongate member (306) to advance under compression; and such that unspooling of feed band (322) may cause retraction of elongate member (306) by increasing the length of service loop (330) to thereby cause elongate member (306) to retract under tension. In some cases, guides (321) may be configured to at least partially nest relative to each other to accommodate decreasing the length of service loop (330) when feed band (322) is spooled.

As also noted above, feed band (322) may be configured to rotate together with elongate member (306) about the roll axis (RA). In this regard, medical system (300) may further include a planar constrainer, such as planar constrainer (202) described above, positioned along service loop (330) and configured to permit movement of service loop (330) within the vertical plane while inhibiting (e.g., preventing) movement of service loop (330) out of the vertical plane. For example, handle adapter (240) may be securely mounted to instrument handle (304), and linkage (242) may be positioned about service loop (330), guides (321), and feed band (322) such that elongate member (306) and feed band (322) each extend through each aperture (254, 259) of links (250, 251). A feed band adapter (not shown) may be incorporated into planar constrainer (202) in lieu of feed adapter (241), and may be securely mounted to the distalmost guide (321) of elongate member feed assembly (320). Thus, planar constrainer (202) may be used in some cases in conjunction with elongate member feed assembly (320).

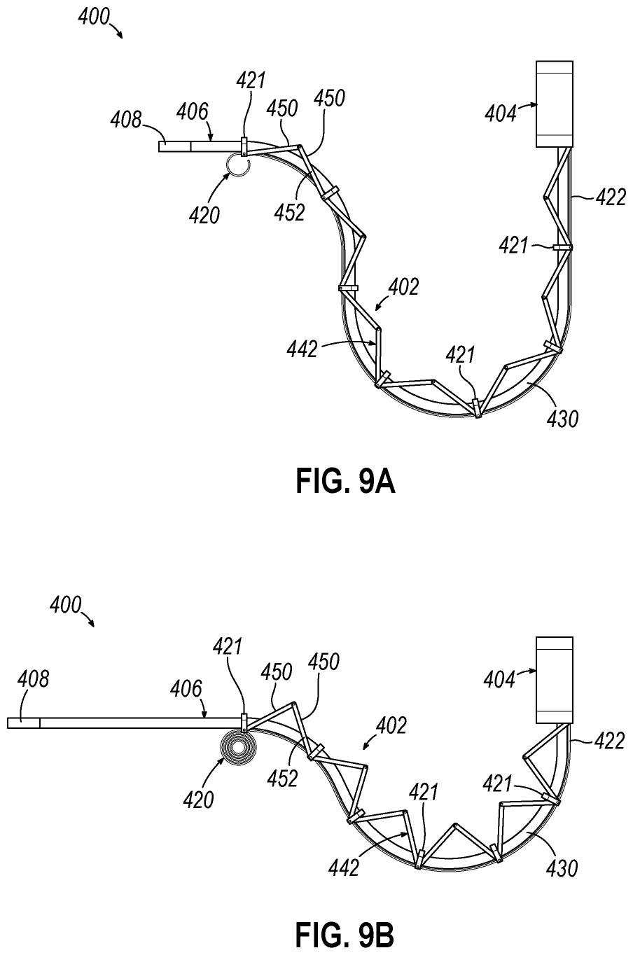

FIGS. 9A-9B depict an example of a medical system (400) having both a planar constrainer (402) and a Bowden-type elongate member feed assembly (420), in accordance with some embodiments.

As illustrated, medical system (400) includes an instrument handle (404) and a flexible elongate member (406) extending distally from instrument handle (404) to a distal end (408). Instrument handle (404) may be used to longitudinally translate (e.g., advance and retract) and/or steer (e.g., articulate) elongate member (406). By way of example only, elongate member (406) may include a scope, a sheath (e.g., an access sheath), a catheter, a shaft of a surgical tool (e.g., a biopsy tool) which may further include an end effector (not shown) at distal end (408) of elongate member (406), and/or any other suitable kind of elongate instrument.

Instrument handle (404) may include at least some drive inputs (not shown) operatively coupled to distal end (408) of elongate member (406) via corresponding pull wires (not shown), such that these drive inputs may be configured to selectively deflect distal end (408) of elongate member (406) laterally and/or vertically away to thereby steer elongate member (406) and/or to facilitate advancement and retraction of at least a proximal portion of elongate member (406). In addition, or alternatively, instrument handle (404) may include one or more drive inputs configured to facilitate rolling of elongate member (406) about a roll axis (RA) extending along a length of elongate member (406), such as in a manner similar to that described above.

In the example shown, medical system (400) also includes elongate member feed assembly (420) positioned distally of instrument handle (404), such as at or near an insertion site of elongate member (406) into the patient, and configured to facilitate advancement and retraction of at least a distal portion of elongate member (406). In this regard, elongate member feed assembly (420) of the present example includes a plurality of guides (421) slidably positioned along a portion of elongate member (406) that is distal of instrument handle (404); and a flexible feed band (422) routed along the same portion of elongate member (406) that guides (421) are positioned along. Feed band (422) may comprise a metallic material, for example. Feed band (422) of the present example is fixedly coupled to instrument handle (404) and slidably received by guides (421), and is configured to spool at or near the insertion site. In this regard, feed band (422) may be configured to be spooled onto a spindle (not shown) at or near the insertion site, and such a spindle may be operatively coupled to a motor (not shown) to facilitate spooling of feed band (422) onto the spindle and unspooling of feed band (422) from the spindle. In some versions, the distalmost guide (421) may be secured against movement relative to the insertion site, such that the distal end of feed band (422) may be stationary during spooling/unspooling of feed band (422). In addition, or alternatively, feed band (422) may be rotationally coupled to elongate member (406) (e.g., via one or more guides (421), such as the proximal-most guide (421)) such that feed band (422) may be configured to rotate together with elongate member (406) about the roll axis (RA).

As shown, a portion of elongate member (406) extends between instrument handle (404) and the distalmost guide (421) of elongate member feed assembly (420), and may have a length greater than the distance between instrument handle (404) and the distalmost guide (421) of elongate member feed assembly (420) to define a service loop (430). As noted above, instrument handle (404) may be configured to advance and retract at least a proximal portion of elongate member (406), while elongate member feed assembly (420) may be configured to advance and retract at least a distal portion of elongate member (406). For example, instrument handle (404) may be configured to advance a portion of elongate member (406) that is proximal of instrument handle (404) into service loop (430), and/or to retract at least a portion of service loop (430); while elongate member feed assembly (420) may be configured to advance at least a portion of service loop (430) into the patient, and/or to retract a portion of elongate member (406) that is distal of elongate member feed assembly (420) into service loop (430). It will be appreciated that providing service loop (430) between instrument handle (404) and elongate member feed assembly (420) may minimize the length of the portion of elongate member (406) that is proximal of instrument handle (404), thereby contributing to the compact configuration of medical system (400).

As shown, service loop (430) may hang (e.g., sag) freely under the influence of gravity between instrument handle (404) and the distalmost guide (421) of elongate member feed assembly (420), such that service loop (430) may reside within a substantially vertical plane (e.g., defined by a line between instrument handle (404) and the distalmost guide of elongate member feed assembly (420)). Since feed band (422) is routed along service loop (430) via guides (421), feed band (422) may reside within the same vertical plane as service loop (430).

As noted above, feed band (422) is configured to spool at or near the insertion site while the proximal end of feed band (422) is configured to be stationary, such that spooling of feed band (422) may cause advancement of elongate member (406) by decreasing the length of service loop (430) to thereby cause elongate member (406) to advance under compression; and such that unspooling of feed band (422) may cause retraction of elongate member (406) by increasing the length of service loop (430) to thereby cause elongate member (406) to retract under tension.

As also noted above, feed band (422) may be configured to rotate together with elongate member (406) about the roll axis (RA). In this regard, medical system (400) of the present example further includes planar constrainer (402) positioned along service loop (430) and configured to permit movement of service loop (430) within the vertical plane while inhibiting (e.g., preventing) movement of service loop (430) out of the vertical plane. In the example shown, planar constrainer (402) includes an expandable linkage (442) extending distally from instrument handle (404) to the distalmost guide (421) of elongate member feed assembly (420).

Linkage (442) of the present example includes a plurality of links (450) pivotably coupled to each other and further pivotably coupled to respective guides (421) of elongate member feed assembly (420). In the example shown, each link (450) includes a plurality of bars (452), and has a substantially rectangular shape. Bars (452) of each link (450) collectively define a corresponding substantially rectangular aperture (not shown) that is elongate in the vertical direction and that is configured to slidably receive elongate member (406). More particularly, each aperture has a width that is substantially equal to or only slightly greater than an external cross-dimension (e.g., diameter) of elongate member (406), such that each aperture is configured to permit sliding of elongate member (406) in a direction substantially transverse (e.g., perpendicular) to the aperture and such that bars (452) are configured to inhibit (e.g., prevent) lateral movement of elongate member (406) within the aperture; and each aperture has a height that is substantially greater than the external cross-dimension (e.g., diameter) of elongate member (406), such that each aperture is also configured to permit sliding of elongate member (406) from one horizontal bar toward the other horizontal bar.

In the example shown, links (450) are arranged in pairs between longitudinally-adjacent pairs of guides (421), with one end of the distal link (450) of each pair pivotably coupled to the distally-adjacent guide (421), and with one of the proximal link (450 of each pair pivotably coupled to the proximally-adjacent guide (421). In some other versions, a middle region of each link (450) may be pivotably coupled to a corresponding guide (421).

Due to the pivotable couplings between adjacent links (450), linkage (442) may be expandable from at least one compressed state to at least one expanded state; and may be compressible from the at least one expanded state to the at least one compressed state. The pivotable couplings between links (450) and guides (421) may allow links (450) to accommodate sliding of guides (421) along elongate member (406) by pivoting in response to such sliding of guides (421); and/or may allow guides (421) to accommodate pivoting of links (450) relative to each other by sliding in response to such pivoting of links (450).

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

A robotic medical system comprising: an instrument handle; a flexible elongate instrument extending distally from the instrument handle for insertion into a patient at an insertion site, wherein the instrument handle is configured to apply torque to the flexible elongate instrument for rolling the flexible elongate instrument about a roll axis, wherein the flexible elongate instrument is configured to hang between the instrument handle and the insertion site to define a service loop; and a planar constrainer extending along the service loop, wherein the planar constrainer is configured to inhibit movement of the service loop out of a plane.

### EXAMPLE 2

The robotic medical system of Example 1, wherein the planar constrainer includes a proximal end configured to be fixedly secured against movement relative to the instrument handle.

### EXAMPLE 3

The robotic medical system of any of Examples 1 through 2, wherein the planar constrainer includes a distal end configured to be fixedly secured against movement relative to the insertion site.

### EXAMPLE 4

The robotic medical system of any of Examples 1 through 3, wherein the planar constrainer is configured to transition between at least one expanded state and at least one compressed state.

### EXAMPLE 5

The robotic medical system of Example 4, wherein the planar constrainer includes an expandable linkage.

### EXAMPLE 6

The robotic medical system of Example 5, wherein the expandable linkage includes a plurality of links pivotably coupled to each other.

### EXAMPLE 7

The robotic medical system of Example 6, wherein each link of the plurality of links includes an aperture configured to slidably receive the service loop.

### EXAMPLE 8

The robotic medical system of Example 7, wherein each aperture has rounded corners.

### EXAMPLE 9

The robotic medical system of any of Examples 6 through 8, wherein the plurality of links includes a plurality of first links and a plurality of second links, wherein each first link is configured to at least partially nest within a corresponding second link when the planar constrainer is in the at least one compressed state.

### EXAMPLE 10

The robotic medical system of any of Examples 1 through 9, wherein the plane is a vertical plane.

### EXAMPLE 11

The robotic medical system of any of Examples 1 through 10, further comprising a feed assembly configured to selectively advance and retract the flexible elongate instrument.

### EXAMPLE 12

The robotic medical system of Example 11, wherein the feed assembly is positioned distal of the instrument handle.

### EXAMPLE 13

The robotic medical system of Example 11, wherein the instrument handle includes the feed assembly.

### EXAMPLE 14

The robotic medical system of any of Examples 11 through 13, wherein the service loop is defined between the instrument handle and at least a portion of the feed assembly.

### EXAMPLE 15

The robotic medical system of any of Examples 11 through 14, wherein the feed assembly includes at least one roller wheel configured to selectively advance and retract the flexible elongate instrument by frictionally engaging the flexible elongate instrument.

### EXAMPLE 16

The robotic medical system of any of Examples 11 through 14, wherein the feed assembly includes a feed band configured to selectively advance and retract the flexible elongate instrument by placing the flexible elongate instrument under at least one of tension or compression.

### EXAMPLE 17

The robotic medical system of Example 16, wherein the feed band is configured to selectively spool and unspool at the instrument handle.

### EXAMPLE 18

The robotic medical system of any of Examples 16 through 17, wherein the feed assembly further includes at least one guide configured to route the feed band along the service loop.

### EXAMPLE 19

The robotic medical system of Example 18, wherein the at least one guide is coupled to the planar constrainer.

### EXAMPLE 20

A device comprising: a proximal portion configured to be fixedly secured against movement relative to an instrument handle of a robotic medical system; a distal portion configured to be fixedly secured against movement relative to an insertion site on a patient; and an expandable linkage extending distally from the proximal portion to the distal portion, wherein the expandable linkage includes a plurality of links pivotably coupled to each other, wherein each link of the plurality of links includes an aperture configured to slidably receive a flexible elongate instrument of the robotic medical system.

### EXAMPLE 21

The device of Example 20, wherein the plurality of links includes a plurality of first links and a plurality of second links, wherein each first link is configured to at least partially nest within a corresponding second link when the expandable linkage is in at least one compressed state.

### EXAMPLE 22

The device of any of Examples 20 through 21, wherein the proximal portion includes a handle adapter configured to mount to the instrument handle of the robotic medical system.

### EXAMPLE 23

The device of any of Examples 20 through 22, wherein the distal portion includes a feed adapter configured to mount to a feed assembly of the robotic medical system.

### EXAMPLE 24

A robotic medical system comprising: the device of any of Examples 20 through 23; and the elongate flexible instrument, wherein the elongate flexible instrument is slidably received by each aperture of the plurality of links.

### EXAMPLE 25

A method comprising: hanging a flexible elongate instrument between an instrument handle and an insertion site on a patient to define a service loop; and constraining, via a planar constrainer, the service loop to movement within a plane.

### EXAMPLE 26

The method of Example 25, further comprising advancing or retracting the flexible elongate instrument, such that the planar constrainer transitions between an expanded state and a compressed state.

### EXAMPLE 27

The method of Example 26, wherein advancing or retracting the flexible elongate instrument is performed via a feed assembly.

### EXAMPLE 28

The method of Example 27, wherein the service loop is defined between the instrument handle and the feed assembly.

### EXAMPLE 29

The method of Example 26, wherein advancing or retracting the flexible elongate instrument is performed manually.

### EXAMPLE 30

The method of any of Examples 26 through 29, further comprising rolling the flexible elongate instrument about a roll axis while the planar constrainer is in at least one of the expanded state or the compressed state.

Use of “or” is intended in the inclusive rather than exclusive sense, unless explicitly stated otherwise or the context clearly dictates otherwise. Thus, for example, reference to “A” or “B” can encompass “A” only, “B” only, or both “A” and “B.” As another example, reference to “A, B, or C” can encompass “A” only, “B” only, “C” only, or any combination of two or more of “A” or “B” or “C.” Accordingly, the term “or” should be generally understood as equivalent to “and/or” unless stated otherwise or the context clearly dictates to the contrary.

It should be appreciated that any specific order of steps shown or described herein is illustrative in nature and should not be construed as required unless explicitly stated or the context clearly dictates otherwise. Thus, for example, with respect to any processes or methods herein, any two or more steps or stages in a method or process may performed serially or in parallel, in any combination, and may be performed in any order, unless explicitly stated or the context clearly dictates otherwise.

In some instances, relative positions or orientations are used, such as top, bottom, upper, lower, forward, backward, front, rear, left, right, up down, horizontal, vertical, longitudinal, lateral, or the like. These terms may be used to refer to an arbitrary frame of reference or a frame of reference shown in the drawings, for purposes of explanation or to demonstrated the relative spatial configurations associated with various elements. These terms should not be understood to require any particular gravitational or other frame of reference explicitly stated or the context clearly dictates otherwise.

To the extent any headings are used through this description, these headings are used for convenience only and should not be construed as limit the scope of disclosure or the description under a heading to only the topic associated with the heading in anyway.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various examples, configurations, or embodiments of the present technology, further adaptations of the systems or methods described herein may be accomplished by appropriate modifications by one of ordinary skill in the art without departing from the scope of the technology described herein. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the claimed subject matter should be considered in terms of the following claims and is understood not to be limited to the details of structure and operation shown and described in the specification and drawings.

1-3. (canceled)

4. A robotic medical system comprising:
an instrument handle;
a flexible elongate instrument extending distally from the instrument handle for insertion into a patient at an insertion site, wherein the instrument handle is configured to apply torque to the flexible elongate instrument for rolling the flexible elongate instrument about a roll axis, wherein the flexible elongate instrument is configured to hang between the instrument handle and the insertion site to define a service loop; and
a planar constrainer extending along the service loop, wherein the planar constrainer is configured to inhibit movement of the service loop out of a plane, wherein the planar constrainer is configured to transition between at least one expanded state and at least one compressed state.

5. The robotic medical system of claim 4, wherein the planar constrainer includes an expandable linkage.

6. The robotic medical system of claim 5, wherein the expandable linkage includes a plurality of links pivotably coupled to each other.

7. The robotic medical system of claim 6, wherein each link of the plurality of links includes an aperture configured to slidably receive the service loop.

8. The robotic medical system of claim 7, wherein each aperture has rounded corners.

9. The robotic medical system of claim 6, wherein the plurality of links includes a plurality of first links and a plurality of second links, wherein each first link is configured to at least partially nest within a corresponding second link when the planar constrainer is in the at least one compressed state.

10-19. (canceled)

20. A device comprising:
a proximal portion configured to be fixedly secured against movement relative to an instrument handle of a robotic medical system;
a distal portion configured to be fixedly secured against movement relative to an insertion site on a patient; and
an expandable linkage extending distally from the proximal portion to the distal portion, wherein the expandable linkage includes a plurality of links pivotably coupled to each other, wherein each link of the plurality of links includes an aperture configured to slidably receive a flexible elongate instrument of the robotic medical system.

21. The device of claim 20, wherein the plurality of links includes a plurality of first links and a plurality of second links, wherein each first link is configured to at least partially nest within a corresponding second link when the expandable linkage is in at least one compressed state.

22. The device of claim 20, wherein the proximal portion includes a handle adapter configured to mount to the instrument handle of the robotic medical system.

23. The device of claim 20, wherein the distal portion includes a feed adapter configured to mount to a feed assembly of the robotic medical system.

24. A robotic medical system comprising:
the device of claim 20; and
the elongate flexible instrument, wherein the elongate flexible instrument is slidably received by each aperture of the plurality of links.

25. (canceled)

26. A method comprising:
hanging a flexible elongate instrument between an instrument handle and an insertion site on a patient to define a service loop;
constraining, via a planar constrainer, the service loop to movement within a plane; and
advancing or retracting the flexible elongate instrument, such that the planar constrainer transitions between an expanded state and a compressed state.

27. The method of claim 26, wherein advancing or retracting the flexible elongate instrument is performed via a feed assembly.

28. The method of claim 27, wherein the service loop is defined between the instrument handle and the feed assembly.

29. The method of claim 26, wherein advancing or retracting the flexible elongate instrument is performed manually.

30. The method of claim 26, further comprising rolling the flexible elongate instrument about a roll axis while the planar constrainer is in at least one of the expanded state or the compressed state.
